SEQUENCE LISTING (16 sequences)

SEQ ID NO: 1 - DNA, 135 bp, Artificial Sequence
  Fragment of exon 12 of the human albumin gene from positions 16283-16423
gctgtcatctcttgtgggctgtaatcatcgtttaagagtaatattgcaaaacctgtcatgcccacacaaatctctccctggcattgttgtctttgcagatgtcagtgaaagagaaccagcagctcccatgagttt

SEQ ID NO: 2 - DNA, 62 bp, Methanobrevibacter smithii
  misc_feature: Fragment of the 16S ribosomal RNA gene
ccgggtatctaatccggttcccgtcagaatcgttccagtcagctcccagggtagaggtgaaa

SEQ ID NO: 3 - DNA, 74 bp, Artificial Sequence
  Fragment of the Methanobrevibacter smithii mcrA gene
gctctacgaccagatmtggcttggargcacckaacamcatggacacwgtccgtagtacgtgaagtcatccagca

SEQ ID NO: 4 - DNA, 65 bp, Methanobrevibacter smithii
  misc_feature: Fragment of the rpoB gene
aagggatttgcacccaacacatttggtaagatttgtccgaatggaccacagttaggaccctctgg

SEQ ID NO: 5 - DNA, 20 bp, Methanobrevibacter smithii
  misc_feature: Primer for the 16S rRNA gene fragment
ccgggtatctaatccggttc

SEQ ID NO: 6 - DNA, 20 bp, Methanobrevibacter smithii
  misc_feature: Primer for the 16S rRNA gene fragment
ctcccagggtagaggtgaaa

SEQ ID NO: 7 - DNA, 22 bp, Methanobrevibacter smithii
  misc_feature: Probe for the 16S rRNA gene fragment
ccgtcagaatcgttccagtcag

SEQ ID NO: 8 - DNA, 24 bp, Methanobrevibacter smithii
  misc_feature: 5′ primer for the mcrA gene fragment
gctctacgaccagatmtggcttgg

SEQ ID NO: 9 - DNA, 25 bp, Methanobrevibacter smithii
  misc_feature: 3′ primer for the mcrA gene fragment
ccgtagtacgtgaagtcatccagca

SEQ ID NO: 10 - DNA, 25 bp, Methanobrevibacter smithii
  misc_feature: Probe for the mcrA gene fragment
argcacckaacamcatggacacwgt

SEQ ID NO: 11 - DNA, 20 bp, Methanobrevibacter smithii
  misc_feature: 5′ primer for the rpoB gene fragment
aagggatttgcacccaacac

SEQ ID NO: 12 - DNA, 22 bp, Methanobrevibacter smithii
  misc_feature: 3′ primer for the rpoB gene fragment
gaccacagttaggaccctctgg

SEQ ID NO: 13 - DNA, 23 bp, Methanobrevibacter smithii
  misc_feature: Probe for the rpoB gene fragment
atttggtaagatttgtccgaatg

SEQ ID NO: 14 - DNA, 22 bp, Artificial Sequence
  5 primer for human albumin
gctgtcatctcttgtgggctgt

SEQ ID NO: 15 - DNA, 23 bp, Artificial Sequence
  3 primer for the human albumin gene fragment
aaactcatgggagctgctggttc

SEQ ID NO: 16 - DNA, 26 bp, Artificial Sequence
  Human albumin probe
cctgtcatgcccacacaaatctctcc

(19) United States
(12) Patent Application Publication
(10) Pub. No.: US 2020/0407779 A1
(43) Pub. Date: Dec. 31, 2020

(54) METHOD FOR DIAGNOSING BACTERIAL VAGINOSIS BY DETECTING METHANOBREVIBACTER SMITHII

(71) Applicants: FONDATION MEDITERRANEE INFECTION, Marseille (FR); UNIVERSITE D'AIX MARSEILLE, Marseille (FR)

(72) Inventors: Didier RAOULT, Marseille (FR); Michel DRANCOURT, Marseille (FR); Florence FENOLLAR, Marseille (FR); Ghiles GRINE, Nouvelle ville Tizi Ouzou (DZ)

(73) Assignees: FONDATION MEDITERRANEE INFECTION, Marseille (FR); UNIVERSITE D'AIX MARSEILLE, Marseille (FR)

(21) Appl. No.: 16/955,187

(22) PCT Filed: Sep. 14, 2018
(86) PCT No.: PCT/FR2018/052260

§ 371 Date: Sep. 2, 2020

(30) Foreign Application Priority Data
Dec. 18, 2017 (FR) 1762319

(51) Int. Cl. C12Q 1/689; C12Q 1/6851; C12Q 1/686

(52) U.S. Cl. CPC C12Q 1/689; C12Q 2531/113; C12Q 1/686; C12Q 1/6851

(57) ABSTRACT

The present invention relates to an in vitro diagnostic method with regard to the presence of bacterial vaginosis, characterized in that the presence of bacterial vaginosis is determined if the archaea Methanobrevibacter smithii is present in a patient vaginal secretion sample by detecting therein the presence of at least one nucleic acid sequence specific to said methanogenic archaea Methanobrevibacter smithii.

### REFERENCE TO SEQUENCE LISTING SUBMITTED VIA EFS-WEB

This application includes an electronically submitted sequence listing in .txt format. The .txt file contains a sequence listing entitled “409545-5_ST25.txt” created on Aug. 29, 2020 and is 4,525 bytes in size. The sequence listing contained in this .txt file is part of the specification and is hereby incorporated by reference herein in its entirety.

The present invention relates to a rapid simplex microbiological diagnosis of bacterial vaginosis (hereinafter abbreviated as “BV”), i.e. by detection of a single microorganism and, if need be, in a non-quantitative manner. More particularly, the present invention relates to a method for diagnosing the state of the vaginal bacterial flora with regard to the presence of bacterial vaginosis (BV), if need be for monitoring the state of the vaginal bacterial flora and its therapeutic management. BV, which is a common infection with harmful consequences for pregnancy and the fetus, has long been defined microbiologically by a near complete disappearance of the normal vaginal flora composed mainly of lactobacilli to the benefit of other bacteria, notably Gardnerella vaginalis, Mobiluncus spp. and genital mycoplasmas [Spiegel C A, CMR 1991; Thorsen P, AJGO 1998]. BV, a common reason for medical consultation, is particularly implicated in susceptibility to sexually transmitted infections such as HIV and in preterm labor and delivery and low birth weight babies. Its prevalence in women, including during pregnancy, is between 8 and 23% [Guise J M, AJPM 2001] according to current methods of investigation.

Currently, the diagnosis of BV is based on the Nugent score and the Amsel criteria. The Nugent score is the most commonly reported method in the literature and is considered by some to be the reference technique, even though it is not routinely performed in clinical microbiology laboratories due to the tedious nature of its implementation [Fredricks DN, NEJM 2005; Thomason J L, AJOG 1992; Ison C A, STD 2002; Nugent R P, JCM 1991]. The Nugent score identifies BV by semi-quantitative morphological analysis of bacteria after Gram staining. It is therefore a subjective technique whose reproducibility has been questioned [Sha B E, JCM 2005; Schwebke J R, OG 1996]. The Amsel clinical criteria (vaginal pH greater than 4.5; adherent homogeneous grayish leucorrhoea; nitrogenous odor after addition of 10% KOH; presence of clue cells) represent the second diagnostic approach [Amsel R AJM 1983]. Like the Nugent score, it is a delicate determination and not used in routine clinical practice.

One of the limitations of these diagnostic methods is the lack of identification of certain microorganisms involved in BV. On the one hand, microscopic detection of microorganisms by Gram staining based on their wall structure, microorganisms without a wall such as mycoplasmas or with a particular wall structure such as archaea, the latter microorganisms cannot be detected by Gram staining and therefore their presence is not taken into account by the Nugent score. On the other hand, the contribution of molecular biology has made it possible to identify new bacteria that may be involved in BV but their detection by the two existing diagnostic methods is impossible. Atopobium vaginae is the main new bacterial species characterized. Its presence has been correlated with BV in a few articles, but a reliable quantitative assessment of its relative place with respect to other microorganisms has not been made [Bradshaw C S, JID 2006, Rodriguez J M, IJSB 1999; Ferris M J, BMCID 2004; Ferris M J, JCM 2004; Verhelst R, BMCM 2004].

In the article published in 2006 by Bradshaw et al. [Bradshaw C S, JID 2006], a relationship was notably described between the detection of A. vaginae and G. vaginalis bacteria and BV, but those results were inadequate to make a reliable diagnosis of BV and/or to reliably monitor the progression of BV. Indeed, the data presented in that work allowed said bacteria to be detected but not truly quantified. Moreover, this screening showed a good sensitivity, A. vaginae and G. vaginalis being detected respectively in 96% and 99% of BV patients. However, its specificity is poor as A. vaginae is detected in 12% of patients with normal flora and G. vaginalis in 60%. The authors then attempted a “semi-quantitative” approach by classifying the bacterial loads as low or high by comparing the median cycle threshold (CT) values for the detection of microorganisms in all samples analyzed. For example, the authors estimated a median load of 4×10⁵ copies for G. vaginalis (median of 21 cycles) and 4×10⁶ copies for A. vaginae (median of 18 cycles). The high loads of G. vaginalis (>4×10⁵) and A. vaginae (>4×10⁶) were significantly more present in BV patients than in patients with normal flora. However, these values still showed poor sensitivity, with A. vaginae and G. vaginalis only detected in 49% and 71% of BV patients, respectively. In addition, 16 patients (28%) with a relapse of BV after treatment had a G. vaginalis concentration below the given threshold (Table 3). Forty patients (70%) with relapsed BV also had a below-threshold concentration of A. vaginae.

The authors' “semi-quantitative” approach is therefore inapplicable as a diagnostic tool for immediate patient monitoring. In fact, the techniques used to obtain these results were inadequate in several respects. First, the PCR techniques were not sufficiently sensitive because the molecular targets amplified excessively long fragments (16S ribosomal RNA fragment of 430 base pairs for A. vaginae and 291 base pairs for G. vaginalis). With such a long targeted sequence, sensitivity in a PCR reaction is low. Furthermore, the real-time PCR techniques used SyberGreen labeling of the amplification product for detection and quantification, which is much less specific than those using labeled hydrolysis probes that require triple specificity (two primers plus the probe, to amplify a fragment no larger than 150 base pairs).

Finally, two of the inventors of the present invention have developed a diagnostic test for multiplex BV based on the molecular detection and quantification of at least the two bacteria Atopobium vaginae and Gardnerella vaginalis in vaginal specimens [Bretelle F, Clin Infect Dis. 2015; 60:860-7; Menard J P., Eur J Clin Microbiol Infect Dis. 2010; 29:1547-52; labor. Obstet Gynecol. 2010; 115:134-40; Clin Infect Dis. 2008; 47:33-43 and WO 2008/062136].

In conclusion, with the exception of the method described in the latter references and WO 2008/062136, current techniques for diagnosing BV are based on unreliable criteria.

However, the implementation of this quantitative, multiplicated diagnostic test of WO 2008/062136 requires a complex set of reagent preparation, performance and interpretation operations.

In particular, in WO 2008/062136, the presence of bacterial vaginosis or the failure of current therapeutic treatment is determined if the concentrations of DNA fragments of the two sequences specific to the bacteria Atopobium vaginae and Gardnerella vaginalis respectively in a patient vaginal secretion sample containing at least 10⁴ human cells/mL are such that at least one of the following two conditions a) and b) is met:
(a) the concentration Ca of said Atopobium vaginae DNA fragment is greater than or equal to 10⁸ copies/mL, and
(b) the concentration Cg of said Gardnerella vaginalis DNA fragment is greater than or equal to 10⁹ copies/mL.

A concentration of Atopobium vaginae bacteria greater than or equal to the threshold of 10⁸ allows the detection of approximately 90% of vaginosis. The quantification of the Gardnerella vaginalis bacterium comes in addition in the case where the concentration of Atopobium vaginae is lower than the threshold of 10⁸ bacteria/mL, to detect vaginosis, because the detection threshold of G. vaginalis greater than or equal to 10⁹ bacteria/mL would only allow the detection of about half of the vaginoses. This is why, according to this method, it is necessary to quantify the DNA concentrations for both bacteria.

On the other hand, it is noted that the development of bacterial vaginosis is confirmed if the concentrations Ca, Cg and CI of at least three fragments of specific sequences present in a single copy in the DNA of the bacteria A. vaginae (Ca), G. vaginalis (Cg) and respectively Lactobacillus sp. (CI) in the DNA extracted from a patient vaginal secretion sample are such that the ratio of concentrations Cl/(Ca+Cg) decreases between the two samples taken successively over time at a sufficient time interval, preferably at least one month.

As used herein, “development of vaginosis” means an aggravation of vaginosis already detected or, in some cases, a risk of developing vaginosis, i.e. an imbalance or abnormality in the vaginal flora that could become pathological.

Similarly, failure of the current treatment based on the concentrations of the specific sequences present in a single copy in the DNA of the bacteria is confirmed if the ratio of concentrations Cl/(Ca+Cg) decreases or does not increase between the two samples taken at a sufficient time interval, preferably at least one month.

More particularly, the concentration of bacteria of the genus Lactobacillus sp. is complementary or confirmatory if the conditions for concentrations of A. vaginae and G. vaginalis are met.

Preferably, a determination of bacterial vaginosis is made if the concentrations are such that the following three conditions are met:

a—concentration Ca in said Atopobium vaginae DNA fragment of specific sequence greater than or equal to 10⁸ copies/mL,

b—concentration Cg in said Gardnerella vaginalis DNA fragment of specific sequence greater than or equal to 10⁹ copies/mL, and

c—concentration CI in said Lactobacillus sp. DNA fragment of specific sequence less than or equal to 10⁷ copies/mL.

Said concentrations Ca, Cg or CI are determined by real-time PCR-type enzymatic amplification and quantification of the DNA of said DNA fragments of sequences specific to the respective bacteria Atopobium vaginae, Gardnerella vaginalis and if need be Lactobacillus sp, as well as, preferably, a human DNA fragment present in any human biological sample containing cells.

The purpose of the present invention is to simplify the implementation of the laboratory diagnosis of BV by providing a simplex (i.e. by detection of a single microorganism) and if possible non-quantitative test.

While continuing to investigate the microorganisms specifically associated with BV, the inventors surprisingly discovered that a methanogenic archaea called Methanobrevibacter smithii, and it alone tested among all the other methanogenic archaea present in humans (explained in Example 2), was detected in 100% of vaginal specimens obtained from patients otherwise diagnosed with BV and in 0% of vaginal specimens obtained from patients without BV according to the reference method by quantification of Atopobium vaginae, Gardnerella vaginalis and if need be Lactobacillus sp. described in WO 2008/062136. Indeed, no other methanogenic archaea has been detected by techniques based on the amplification and sequencing of the 16S ribosomal RNA gene, techniques which allow the detection of any species of methanogenic archaea.

This result was unexpected because although the detection of this methanogenic archaea had been reported in 1990 in two vaginal specimens collected from two patients with BV, the vaginal specimens collected from another BV patient did not show archaea in this publication [Belay N, J Clin Microbiol. 1990; 28:1666-8]. This work was not only limited in the number of specimens studied, but was also limited in the method of identification, which was purely phenotypic by simple visual observation of the colonies used and did not give a 100% positive association to BV in terms of specificity, and did not allow the use of M. smithii detection as a diagnostic test for BV.

The inventors have demonstrated according to the present invention the possibility of a reliable, rapid and simplified microbiological diagnosis of BV, in particular simple to implement routinely in clinical microbiology laboratories and at the point-of-care [Drancourt M, in Clinical Microbiology. Clin Microbiol Rev. 2016 July; 29(3):429-47].

To do so, the inventors expanded previous studies of the microbial flora of the BV to the study of methanogenic archaea by molecular and culture detection methods.

In the prior art, the different methanogenic archaea detected in humans have been detected in the microbiota of the digestive tract and the oral cavity. Among these 15 species of methanogenic archaea, only 7 have been isolated and cultured in humans (as explained in Table A of Example 2).

This study, reported in the examples below, unexpectedly showed that a methanogenic Methanobrevibacter smithii archaea could be present in vaginal specimens and that only vaginal specimens from patients with BV carried a Methanobrevibacter smithii methanogenic archaea, whereas the archaea M. smithii was never detected in vaginal specimens from BV-free women; and that specific sequence detection of certain single-copy genes such as the mcrA, rpoB and 16S RNA genes of Methanobrevibacter smithii could provide a simplex, non-quantitative diagnostic test for the presence of M. smithii in a vaginal cavity specimen. Thus, according to the present invention it has been demonstrated that the presence of Methanobrevibacter smithii is associated with BV in a very specific and significant manner and that any detection of this archaea makes diagnosis easy and reliable.

More precisely, the present invention provides a method for the in vitro diagnosis of the presence of bacterial vaginosis in a patient, characterized in that the following steps are carried out wherein:
a) the total DNA contained in the vaginal secretion sample is extracted by a method capable of extracting DNA from methanogenic archaea preferably comprising at least two steps of enzymatic and/or mechanical DNA lysis, and
b) the presence of bacterial vaginosis is determined if the DNA extracted from said vaginal secretion sample of said patient is detected to contain said sequence specific to human DNA and at least one nucleic acid sequence specific to said methanogenic archaea Methanobrevibacter smithii, the concentrations of the DNA fragments of said sequence specific to human DNA and said sequence specific to Methanobrevibacter smithii in said vaginal secretion sample each being greater than or equal to 10¹ copies/mL.

This 10-copy threshold was determined by comparative tests with a dilution range as explained in Example 1 below.

The present invention therefore allows in vitro diagnosis and monitoring of the state of the vaginal bacterial flora with regard to the presence of bacterial vaginosis, and if need be for the monitoring of its therapeutic treatment, by detecting the presence of the methanogenic archaea Methanobrevibacter smithii alone.

In particular, the presence of Methanobrevibacter smithii is determined by detecting the presence of at least one nucleic acid sequence specific to said Methanobrevibacter smithii methanogenic archaea present in a single copy in said Methanobrevibacter smithii archaea contained in the DNA extracted from said vaginal secretion sample of said patient, said sequence specific to Methanobrevibacter smithii having a size of less than 150 nucleotides.

More particularly, in step b), the following steps are carried out:

b.1) PCR-type enzymatic amplification of at least one said sequence specific to said methanogenic archaea Methanobrevibacter smithii is performed in the DNA extracted from said vaginal secretion sample, and

b.2) amplified fragments of said sequence specific to Methanobrevibacter smithii are detected preferably by sequencing, by agarose gel electrophoresis or by using labeled probes specific for said sequence specific to said Methanobrevibacter smithii methanogenic archaea of sequences distinct from those of said amplification primers.

More particularly, a method is carried out wherein: a specific protocol is carried out for extracting the total DNA contained in the vaginal sample. This extraction protocol may be a simplified protocol including the extraction of DNA from Methanobrevibacter smithii, which is thick-walled and difficult to extract DNA from, or it may be a standard protocol previously described as disclosed in Examples 1 to 3.

More particularly, a method of extraction of the total DNA contained in the vaginal sample is carried out, including the extraction of DNA from Methanobrevibacter smithii, wherein only the following steps are carried out:

1) mechanical lysis of said sample of vaginal secretions is carried out, preferably by sonication, in particular by an ultrasound sonicator, for example a Branson 2510 sonicator (Branson, Rungis, France), power 4 (i.e. at maximum power), at 50% of the active cycle (i.e. for 30 seconds), and

2) an enzymatic lysis of said sample is carried out, in particular using a DNA extractor such as the Qiagen EZ1 XL apparatus and the Qiagen EZ1® DNA Tissue Kit.

Preferably, said specific sequence of said methanogenic archaea Methanobrevibacter smithii has a size of 70 to 150 nucleotides, preferably 90 to 120 nucleotides.

Preferably still, amplification and quantification reactions are carried out by real-time PCR, using hydrolysis probes specific respectively for each of said specific sequences of said bacteria and specific sequence of a human gene present in any biological sample containing human cells, in the sample to be tested.

The real-time PCR technique consists of conventional PCR using forward and reverse primers and includes detection of the amplified product based on the measurement of fluorescence emission proportional to the quantity of genes amplified with a so-called “hydrolysis” probe. For this purpose, said probe is 5′-labeled with a fluorescence emitter or fluorophore and 3′-labeled with a fluorescence emission blocking agent. This blocking agent absorbs the fluorescence emitted when the fluorophore and the blocking agent are close together. When the fluorophore and the blocking agent are separated, the fluorescence emission is no longer absorbed by the blocking agent. As the Taq polymerase passes through, it causes hydrolysis of the probe and thus release of the nucleotides and fluorophore in solution. The fluorescence emission will therefore be proportional to the number of amplifiers. The principle of real-time PCR is based on the ability of the Taq polymerase during the elongation step to hydrolyze a probe hybridized to the DNA to be copied, this hydrolysis allowing the emission of fluorescence, which allows quantification. During the same reaction, two different targets can be quantified by introducing into the reaction mixture two primers and a probe directed against one target, and two other primers and a probe directed against the other target. The two probes are labeled with different fluorophores.

“Sequence specific to said archaea” means a sequence of the genome of said archaea which is not found in any other genome of a living organism.

“DNA fragment” means a DNA fragment or oligonucleotide whose sequences are written below in the 5′-3′ direction.

More particularly, reactions are carried out to amplify and quantify a sequence specific to human DNA in the test sample, comprising a sequence specific to human albumin.

More particularly, said sequence specific to said methanogenic archaea Methanobrevibacter smithii comprises or is comprised in one of the following fragments:
the fragment from positions 334632 to 334687 of the 16S ribosomal RNA gene with GenBank accession number NC_009515.1.
the fragment from positions 1734995 to 1735069 of the mcrA gene of the protein methyl coenzyme M reductase alpha subunit of 157 amino acids and 16.942 Da with GenBank accession number AAW80308.1.
the fragment from positions 876305 to 876370 of the rpoB gene of the M. smithii RNA polymerase subunit B protein of 186 amino acids and 20.836 Da with GenBank accession number ABYV02000000.

More particularly, said sequence specific to human DNA in the test sample comprises the fragment from positions 16283-16423 of exon 12 of the human albumin gene with GenBank accession number M12523.1.

Specifically, in step b), the following steps are implemented wherein:

b.1) a PCR-type enzymatic amplification reaction is carried out on the DNA of at least one said sequence specific to said methanogenic archaea Methanobrevibacter smithii, in the DNA extracted from said samples to be tested, using at least one set of primers capable of amplifying said sequence specific to said methanogenic archaea Methanobrevibacter smithii, and

b.2) it is checked whether the possible amplifiers of the DNA extracted from said samples to be tested comprise a said specific sequence using a hydrolysis probe comprising a sequence specific to said sequence specific to Methanobrevibacter smithii and flanked by the sequences of said primers.

More particularly, co-amplification and quantification reactions are carried out using two sets of primers and hydrolysis probes specific respectively on the one hand to said sequence specific to the archaea Methanobrevibacter smithii, and on the other hand to a sequence specific to human DNA in the sample to be tested, preferably a sequence specific to human albumin, and said sequence specific to human DNA comprising a sequence of said probe flanked by sequences suitable for use as said primers in a PCR-type amplification reaction of said sequence specific to human DNA.

More particularly still, the presence of bacterial vaginosis is determined if, in the DNA extracted from a patient vaginal secretion sample, the following two conditions a) and b) are met:

a) the concentration Ca of the human albumin DNA fragment is greater than or equal to 10¹ copies/mL, and

b) the concentration Cm of said DNA fragment specific to Methanobrevibacter smithii is greater than or equal to 10¹ copies/mL.

More particularly, said sequence specific to said methanogenic archaea Methanobrevibacter smithii is selected from the following sequences including probe sequences (underlined) flanked by primer sequences (in bold) or their reverse and complementary sequences for antisense primers:

-for the 16S rRNA sequence:
SEQ ID NO: 2 =
5′-CCGGGTATCTAATCCGGTTCCCGTCAGAATCGTTCC
AGTCAGCTCCCAGGGTAGAGGTGAAA-3′ (this sequence SEQ
ID NO: 2 was described in the article by Dridi B,
PLoS One 4(9):e7063);
-for the mcrA gene sequence:
SEQ ID NO: 3 =
5′-GCTCTACGACCAGATMTGGCTTGGARGCACCKAACAMCATGGACACW
GTCCGTAGTACGTGAAGTCATCCAGCA-3′ (this sequence SEQ
ID NO: 3 was described in the article by Vianna
Oral Microbiology and Immunology, 24(5), pp.
417-422).
-for the rpoB sequence:
SEQ ID NO: 4 =
5′-AAGGGATTTGCACCCAACACATTTGGTAAGATTTGTCCGAATG
GACCACAGTTAGGACCCTCTGG-3′ (this sequence SEQ ID
NO: 4 was described in the article by Dridi B,
PLoS One 4:e7063).

In the writing of these sequences, the broad specificity of certain enzymes or the degeneracy of the genetic code implies that letters corresponding to several different nitrogenous bases for the same nucleotide present at a position must sometimes be indicated in the sequences. In the case in our mcrA sequence: R corresponds to a purine either A or G; K (keto) corresponds to G or T; M corresponds to A or C and W (weak) corresponds to A or T.

Further advantageously, amplification and quantification reactions are carried out using primer sets and hydrolysis probes specific to said archaea Methanobrevibacter smithii, and if need be to a sequence specific to human DNA in the sample to be tested, such as a sequence specific to human albumin, and said specific sequence comprises a probe sequence flanked by sequences suitable for use as a primer in a PCR-type amplification reaction of said specific sequences.

As used herein, “probe” means an oligonucleotide, preferably still 20 to 30 nucleotides, which specifically hybridizes to said specific sequence and thus makes it possible to detect and quantify it specifically by measuring the increase in fluorescence binding of the PCR reaction.

The probe allows the amplified specific DNA to be detected and quantified.

As used herein, “primer” means an oligonucleotide preferably of 15 to 25 nucleotides that specifically hybridizes to one of the two ends of the sequence that the DNA polymerase will amplify in the PCR reaction.

More particularly, said sequence of exon 12 of the human albumin gene specific to human DNA in the test sample comprises the following sequence of the sequence listing or the complementary sequence:

SEQ ID NO: 1 =
5′-GCTGTCATCTCTTGTGGGCTGTAATCATCGTTTAAGAGTAATATTG
CAAAACCTGTCATGCCCACACAAATCTCTCCCTGGCATTGTTGTCT
TTGCAGATGTCAGTGAAAGAGAACCAGCAGCTCCCATGAGTTT-3′

More particularly still, the primers and probes sets chosen, if need be, from the following sequences of the sequence listing appended to the present description, or their complementary sequences, are used:

-for the 16S rRNA gene:
5′ primer:
SEQ ID NO: 5 =
5′-CCGGGTATCTAATCCGGTTC-3′
3′ primer:
SEQ ID NO: 6 =
5′-CTCCCAGGGTAGAGGTGAAA-3′
Probe:
SEQ ID NO: 7 =
5′-CCGTCAGAATCGTTCCAGTCAG-3′
-for the mcrA gene:
5′ primer:
SEQ ID NO: 8 =
5′-GCTCTACGACCAGATMTGGCTTGG-3′
3′ primer:
SEQ ID NO: 9 =
5′-CCGTAGTACGTGAAGTCATCCAGCA-3′
Probe:
SEQ ID NO: 10 =
5′-ARGCACCKAACAMCATGGACACWGT-3′
-for the rpoB gene:
5′ primer:
SEQ ID NO: 11 =
5′-AAGGGATTTGCACCCAACAC-3′
3′ primer:
SEQ ID NO: 12 =
5′-GACCACAGTTAGGACCCTCTGG-3′
Probe:
SEQ ID NO: 13 =
5′-ATTTGGTAAGATTTGTCCGAATG-3′
-for human albumin:
5′ primer:
SEQ ID NO: 14 =
5′-GCTGTCATCTCTTGTGGGCTGT-3′
3′ primer:
SEQ ID NO: 15 =
3′-AAACTCATGGGAGCTGCTGGTTC-3′
Probe:
SEQ ID NO: 16 =
5′-CCTGTCATGCCCACACAAATCTCTCC-3′

The present invention also relates to a diagnostic kit useful for the implementation of a method for diagnosing vaginosis according to the invention, characterized in that it comprises at least:
reagents for extracting the total DNA contained in a vaginal sample, and
a set of primers specific for at least one DNA sequence specific to said methanogenic archaea Methanobrevibacter smithii and preferably at least one probe specific for said sequence specific to said methanogenic archaea Methanobrevibacter smithii, and
reagents for carrying out a PCR-type DNA amplification reaction.

More particularly, a diagnostic kit according to the invention includes:
at least one set of primers and probes of sequences selected from the 3 sets of sequences SEQ ID NO: 5 to 7, SEQ ID NO: 8 to 10 and SEQ ID NO: 11 to 13, and
at least one set of primers and probe of sequence SEQ ID NO: 14 to 16.

The sequences SEQ ID NO: 1 to 16 described above are specified in the sequence listing appended to the present description.

At the position corresponding to a nucleotide K, M, R, W or T in the sequences SEQ ID NO: 3, 8 and 10, variable nucleotides as defined above are found in the complementary target sequences.

The oligonucleotides of SEQ ID NO: 3, 8 and 10, are thus implemented, in fact, in the form of equimolar mixtures of oligonucleotides of different sequences, said oligonucleotides of different sequences responding for each sequence SEQ ID NO: 3 8 and 10 to the various possible definitions of the sequences respectively NO: 3 8 and 10, namely:
an equimolar mixture of 32 different sequences for SEQ ID NO: 3 for which M (present twice) is A or C each time, R is A or G, K is G or T and W is A or T,
an equimolar mixture of 2 different sequences for SEQ ID NO: 8 for which M is A or C,
an equimolar mixture of 16 different sequences for SEQ ID NO: 10 for which M is A or C, R is A or G, K is G or T and W is A or T.

These equimolar mixtures of oligonucleotides are obtained by using, during oligonucleotide synthesis, equimolar mixtures of the different nucleotides concerned.

Other features of the present invention will appear in the light of the detailed description that will follow from the example embodiment with reference to the sequence listing and to the following figures, wherein:

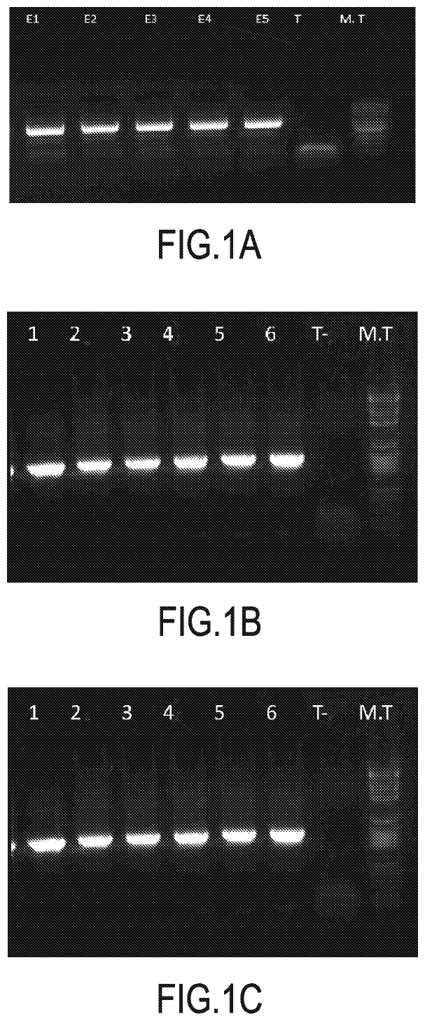

FIGS. 1A to 1C represent the detections by agarose gel electrophoresis of the PCR amplification products of the Methanobrevibacter smithii 16S rRNA gene (FIGS. 1A and 1B) and the Methanobrevibacter smithii mcrA gene (FIG. 1C) in vaginal samples, in the presence of negative controls, by a simplified DNA extraction method according to the present invention in Example 1, and

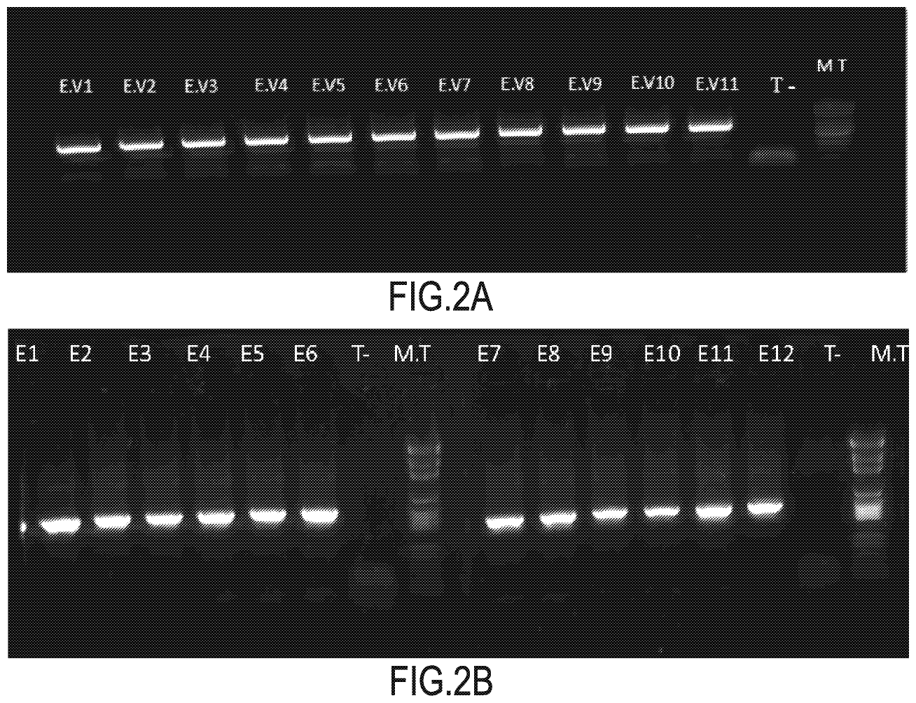

FIGS. 2A and 2B represent the detections by agarose gel electrophoresis of the PCR products of the Methanobrevibacter smithii 16S ribosomal RNA gene (FIG. 2A) and the Methanobrevibacter smithii mcrA gene (FIG. 2B) in vaginal samples, in the presence of negative controls, according to a standard DNA extraction method according to the present invention in Example 2.

### EXAMPLE 1. RAPID EXTRACTION OF METHANOBREVIBACTER SMITHII DNA FOR STANDARD PCR AMPLIFICATION FROM VAGINAL SPECIMENS

In this example, DNA extraction was performed from a suspension of Methanobrevibacter smithii calibrated to 10² colony-forming units (CFUs) according to the protocol below.

A first sonication step was performed for 30 minutes using the Branson 2510 ultrasonic sonicator (Branson, Rungis, France) power 4, at 50% of the active cycle, followed by a second step of enzymatic lysis of the wall of Methanobrevibacter smithii and DNA purification using the V 1.066069118 Qiagen DNA bacteria card contained in the Qiagen EZ1 XL device and the Qiagen EZ1® DNA Tissue Kit following the supplier's instructions (Qiagen, Les Ulis, France). In this example, a portion of the Methanobrevibacter smithii 16S ribosomal gene was amplified by standard PCR from vaginal specimens using a simplified total DNA extraction method, as explained in this example.

The polymerase chain reactions (PCRs) were performed in an automatic thermal cycler PTC-200 (MJ Research, Waltham, Mass., USA) including 50 μL of PCR MIX (mixture) comprising: 25 μL of amplification reagent “amplitaq gold” (Thermo Fisher Scientific, Villebon sur Yvette, France); 17 μL of RNase-free distilled water (Sigma Aldrich, Saint-Quentin-Fallavier, France); sense (5′) primer 20 μM 1.5 μL; reverse primer 20 μM 1.5 μL and 5 μL of extracted DNA. The PCR program depends on the primers used. For the amplification of the 16S RNA archaea gene the program comprises: a 1st step at 95° C. for 15 minutes; 3 steps of 40 cycles 95° C. for 30 seconds, 57° C. for 45 seconds, 72° C. for 1 minute; and a final step 72° C. for 5 minutes. For the methanogenic mcrA archaea gene, the program comprises: a 1st step of 95° C. for 15 minutes; 03 steps of 40 cycles 95° C. for 1 minute, 57° C. for 45 seconds, 72° C. for 1 minute; and a last step 72° C. for 5 minutes. The PCR products are then migrated onto a 1.5% agarose gel (BIO-RAD, Marnes-la-Coquette, France) for 20 minutes at 135 volts. To confirm that it is Methanobrevibacter smithii, amplification sequencing is performed as follows: sequencing reactions are performed using the “BigDye Terminator v1.1” sequencing kit according to the manufacturer's instructions (Applied Biosystems, Foster City, Calif., USA). All PCR products were sequenced in both directions, using the same primers as those used for PCR, in an automatic PTC-200 thermal cycler (MJ Research, Waltham, Mass., USA) with an initial denaturation step of 1 min at 96° C. followed by 25 cycles of 10 seconds at 96° C., 5 seconds at 50° C. and 3 minutes at 60° C. The sequenced products were purified using 96-well Millipore MultiScreen plates (Merck, Molsheim, France) containing 5% Sephadex G-50 (Sigma-Aldrich, L'Isle d'Abeau Chesnes, France). The sequences are then analyzed on an ABI PRISM 31309 genetic analyzer (Applied Biosystems, Foster City, USA). After all PCR products have been sequenced using the ChromasPro software (http://technelysium.com.au/wp/chromaspro/) the different fragments are assembled and compared to the sequences available in the GenBank database using the NCBI online BLAST program.

The results show that the extracted DNA is of sufficient quality to detect M. smithii by PCR.

FIG. 1A shows a 1.5% agarose gel electrophoresis in which standard PCR amplification products were migrated from five samples E1 to E5 positive for the presence of Methanobrevibacter smithii showing a band with an expected molecular weight of 700 base pairs, in the presence of a negative control which remained negative. The right column labeled “M. size” corresponds to the molecular weight marker. Next, to measure the sensitivity of this new DNA extraction protocol, the inventors tested this protocol on different concentrations of Methanobrevibacter smithii (from 10¹ to 10⁶ CFU/mL. FIGS. 1B and 1C show two 1.5% agarose gels in which the amplification products of different concentrations of Methanobrevibacter smithii DNA were migrated using standard PCR. The amplified products obtained show a band with an expected molecular weight of 700 base pairs for the 16S ribosomal RNA gene amplification product and a band with an expected molecular weight of 560 base pairs for the mcrA gene amplification product, in the presence of a negative control which remained negative. The right column labeled “M. size” corresponds to the molecular weight marker.

FIG. 1B shows the detection of PCR products targeting the Methanobrevibacter smithii 16S ribosomal RNA gene by 1.5% agarose gel electrophoresis. Lanes 1 through 6 correspond to 10¹-10⁶ CFUs of Methanobrevibacter smithii; T−: Negative control; and MT: size marker.

FIG. 1C shows the detection of PCR products targeting the Methanobrevibacter smithii mcrA gene by 1.5% agarose gel electrophoresis. Lanes 1 to 6 correspond to 10¹-10⁶ CFUs of Methanobrevibacter smithii; T−: Negative control; and MT: size marker.

### EXAMPLE 2: COMPARATIVE DNA EXTRACTION TESTS

M. smithii has an extremely strong cell wall that is poorly lysed by the DNA extraction protocols used in routine diagnostics, which explains the failure of routine molecular detection of M. smithii. Alternative protocols for lysis of the M. smithii cell wall should be implemented as one of the two protocols respectively presented in the following comparative trials.

Three methods of DNA extraction from methanogens were tested on 10 suspensions of Methanobrevibacter smithii at 10³ CFU and on 10 human stool samples. The 3 methods were performed as follows:

Method 1: The automated protocol involves the extraction of DNA using the EZ1 Advanced XL extractor with the V 1.066069118 Qiagen DNA card and following the indications of the EZ1® DNA tissue kit (Qiagen, Courtaboeuf, France) described by the manufacturer.

Method 2: The manual DNA extraction protocol uses the “NucleoSpin Tissue Mini Kit” (Macherey-Nagel, Hoerdt, France) following the following steps: 0.3 g of glass powder (B106 mm, Sigma, Saint-Quentin Fallavier, France) is added to 250 μL of sample followed by mechanical lysis in a FastPrep BIO 101 apparatus (Qbiogene, Strasbourg, France) for 2 min at a speed of 6.5. Next, 200 μL of lysis buffer and 20 μL of Proteinase K (20 mg/mL) are added to the samples which are then incubated for 12 hours at 56° C. After 12 hours of incubation, another mechanical lysis is performed at 6.5 speed for 2 minutes. The recovered lysate is processed according to the manufacturer's recommendations. The DNA is eluted in 100 μL of elution buffer and stored at −20° C.

Method 3: A first sonication step was performed for 30 minutes using the Branson 2510 ultrasound sonicator (Branson, Rungis, France) power 4, at 50% of the active cycle, followed by a second step using the automated protocol: EZ1 DNA extractor with the V 1.066069118 Qiagen DNA bacteria card and the Qiagen EZ1® DNA Kit (Qiagen, Courtaboeuf, France).

The 3 methods were compared according to the concentrations of DNA present in the samples, measured using the ThermoSCIENTIFIC 2000 nanodrop assay and a quantitative PCR targeting M. smithii 16S rRNA.

### Result

TABLE 1
M. smithii DNA concentrations obtained with the 3 methods
measured with NANODROP 2000

| | DNA concentrations ng/μL | | |
|---|---|---|---|
| Sample No. | Method 1 | Method 2 | Method 3 |
| 1 | 5 | 22 | 19 |
| 2 | 10 | 32 | 36 |
| 3 | 12 | 31 | 47 |
| 4 | 13 | 26 | 33 |
| 5 | 9 | 28 | 46 |
| 6 | 6 | 25 | 23 |
| 7 | 8 | 27 | 35 |
| 8 | 15 | 42 | 22 |
| 9 | 12 | 28 | 48 |
| 10 | 13 | 33 | 32 |
| Average | 10.3 | 29.4 | 34.1 |
| (ng/μL) | | | |

TABLE 2
M. smithii qPCR results obtained with the 3 methods on
M. smithii suspensions.

| | results obtained with qPCR (Ct) on M. smithii suspension | | |
|---|---|---|---|
| Sample No. | Method 1 | Method 2 | Method 3 |
| 1 | 0 | 32.56 | 30.45 |
| 2 | 0 | 32.08 | 30.07 |
| 3 | 39.56 | 30.54 | 28.45 |
| 4 | 37.56 | 30.65 | 31.65 |
| 5 | 0 | 29.09 | 27.86 |
| 6 | 0 | 29.57 | 32.65 |
| 7 | 0 | 31.54 | 29.76 |
| 8 | 37.09 | 33.43 | 33.65 |
| 9 | 0 | 30.43 | 28.76 |
| 10 | 0 | 30.45 | 30.02 |
| average (ct) | >38 | 31.034 | 30.32 |

TABLE 3
M. smithii qPCR results obtained with the 3 methods on
stool samples.

| | results obtained with qPCR (Ct) on stool | | |
|---|---|---|---|
| | samples | | |
| Sample No. | Method 1 | Method 2 | Method 3 |
| 1 | 0 | 39.65 | 37.05 |
| 2 | 0 | 34.9 | 34.56 |
| 3 | 0 | 38.87 | 37.45 |
| 4 | 0 | 39.56 | 39.89 |
| 5 | 0 | 37.78 | 33.12 |
| 6 | 0 | 40.65 | 38.45 |
| 7 | 42.65 | 38.76 | 37.56 |
| 8 | 0 | 0 | 0 |
| 9 | 0 | 0 | 0 |
| 10 | 0 | 0 | 0 |
| average (ct) | >42 | 27.17 | 25.80 |

Interpretation: Method 3 combining mechanical lysis by sonication followed by enzymatic lysis is the method with the highest extraction yield, Method 2 with two mechanical lysis methods is an intermediate method and Method 1 with only one enzyme lysis cannot be validly used.

### EXAMPLE 3: DETECTION OF SEQUENCES SPECIFIC TO METHANOBREVIBACTER SMITHII IN PATIENT VAGINAL SPECIMENS BY AMPLIFICATION AND SEQUENCING

A total of 77 of the pregnant women, followed up for their pregnancy, were recruited at the La Conception hospital in Marseille. Informed consent is the necessary condition for inclusion. Samples were taken from the posterior vaginal cul-de-sac under sterile, unlubricated speculum and without antiseptic. Four samples were taken from each woman: two cotton specimen samples on a dry tube (Copan Innovation®, Brescia, Italy) and two cytobrush samples (Scrinet® 5.5 mm, laboratoire C.C.D. international, Paris, France). A standard cotton specimen is used for fresh and bacterial culture. A second is placed in a specific transport medium (R1 Urea-Arginine LYO 2, BioMérieux SA, Marcy l'Etoile, France) for the detection of genital mycoplasmas (M. hominis and M. urealyticum). A cytobrush is used for slide staining and Gram staining. A second for DNA extraction for molecular amplification is transported in 500 μL of MEM transport medium (Minimum Essential Medium, Invitrogen Life Technologies, Carlsbad, Calif., USA). It is frozen at −80° C. from its arrival in the laboratory until its use. After appropriate microbiological analyses as previously described in patent WO 2008/062136], 15 patients were diagnosed with bacterial vaginosis according to the criteria recalled in this patent and 62 patients were diagnosed without bacterial vaginosis.

Molecular detection of sequences specific to the methanogenic archaea M. smithii was performed by standard PCR after extraction of the DNA according to a standard protocol. In practice, the DNA extraction protocol uses the “NucleoSpin Tissue Mini Kit” (Macherey-Nagel, Hoerdt, France) modified as follows: 0.3 g of glass powder (B106 mm, Sigma, Saint-Quentin Fallavier, France) is added to 250 μL of sample followed by mechanical lysis in a FastPrep BIO 101 apparatus (Qbiogene, Strasbourg, France) for 2 min at a speed of 6.5. Next, 200 μL of lysis buffer and 20 μL of Proteinase K (20 mg/mL) are added to the samples which are then incubated for 12 hours at 56° C. After 12 hours of incubation, another mechanical lysis is performed at 6.5 speed for 2 minutes. The recovered lysate is processed according to the manufacturer's recommendations. The DNA is eluted in 100 μL of elution buffer and stored at −20° C.

The analysis of data from the literature, and sequences deposited in the “GenBank” site (http://www.ncbi.nlm.nih.gov/Genbank/GenbankSearch.html), makes it possible to see the sequences available for each of the target microorganisms. The specificity of the primers and of fragments of the target sequences of each of the selected microorganisms are tested for their specificity on the NCBI website (http://WWW.ncbi.nlm.nih.gov/BLAST/).

The inventors selected targets on the methanogenic archaea Methanobrevibacter smithii. The selected targets are located on the sequence of the gene coding for 16S ribosomal RNA, on the mcrA gene and on the rpoB gene. A sequence located in exon 12 of the human albumin gene is selected to attest to the presence and amount of DNA in the test sample.

For Methanobrevibacter smithii and human albumin, a probe and a pair of sense and antisense primers are chosen on the previously defined target sequences using the Primer 3® program (http://frodo.wi.mitedu/primer3/primer3_code.html). The primers are described below. Each primer is analyzed on the NCBI website (http://WWW.ncbi.nlm.nih.gov/BLAST/) to ensure their in silico specificity.

-For the 16S rRNA gene:
5′ primer:
SEQ ID NO: 5 =
5′-CCGGGTATCTAATCCGGTTC-3′
3′ primer:
SEQ ID NO: 6 =
5′-CTCCCAGGGTAGAGGTGAAA-3′
-For the mcrA gene:
5′ primer:
SEQ ID NO: 8 =
5′-GCTCTACGACCAGATMTGGCTTGG-3′
3′ primer:
SEQ ID NO: 9 =
5′-CCGTAGTACGTGAAGTCATCCAGCA-3′.
-For the rpoB gene:
5′ primer:
SEQ ID NO: 11 =
5′-AAGGGATTTGCACCCAACAC-3′
3′ primer:
SEQ ID NO: 12 =
5′-GACCACAGTTAGGACCCTCTGG-3′
-For human albumin:
5′ primer:
SEQ ID NO: 14 =
5′-GCTGTCATCTCTTGTGGGCTGT-3′
3′ primer:
SEQ ID NO: 15 =
3′-AAACTCATGGGAGCTGCTGGTTC-3′

In order to test the specificity of the primers and the PCR protocol, DNA is extracted from reference bacterial strains representative of the flora of the vaginal cavity according to the following list: Bacteroides nordii, Propionibacterium avidum, Clostridum irregular, Clostridum massilioamazoniensis, Clostridum butyricum, Clostridum beijerinckii, Bacteroides thetaiotaomicron, Propionibacterium acnes, Finegoldia magna, Bacteroides fragilis, Staphylococcus aureus, Enterobacter aerogenes, Escherichia coli, Klebsiella oxytoca, Streptococcus agalactiae, Serratia marcescens, Enterococcus faecalis, Proteus mirabilis, Pseudomonas aeruginosa, Streptococcus mitis, Staphylococcus epidermidis, Morganella morganii, Citrobacter freundii, Enterobacter cloacae, Bacillus circulans, Neisseria meninigitidis, Streptococcus pneumoniae, Staphylococcus hominis, Acinetobacter baumanii, Haemophilus influenzae was used to perform PCR to confirm the specificity of the primers used.

All negative controls tested are PCR negative. Of the 77 specimens analyzed, 62 specimens corresponding to specimens from normal vaginal flora are PCR negative and 15 specimens corresponding to vaginosis specimens are PCR positive. PCR is followed by amplification sequencing. Analysis of the sequences obtained showed 100% identity with the homologous fragment of the reference Methanobrevibacter smithii 16S ribosomal RNA gene strain NVD (NCBI accession: LT223565) confirming that only this methanogenic archaea was found in vaginal samples taken from pregnant women diagnosed with bacterial vaginosis (FIG. 2A, FIG. 2B and Table 1). Indeed, there are currently 15 methanogenic archaea listed in Table A below detected in humans, of which 7 species (in bold in Table A) are cultured. It was therefore necessary to use a sequencing method that would make it possible to unambiguously identify that, among these 15 species, only the methanogenic archaea Methanobrevibacter smithii was detected.

TABLE A

| Species of methanogenic | Detection | |
|---|---|---|
| archaea | techniques | Sources |
| Methanobrevibacter smithii | culture | stool, oral cavity |
| methanobrevibacter oralis | culture | stool, oral cavity |
| Methanosphaera stadtmanae | culture | stool |
| Methanomassilicoccus | culture | stool |
| luminyensis | | |
| Ca. Methanomassillicoccus | culture | oral cavity |
| intestinalis | | |
| Ca. Methanomethylophilus alvus | culture | stool |
| Methanobrevibacter arbophilus | culture | stool |
| Methanosarcina mazei | molecular biology | oral cavity |
| Ca. Methanomethylophilus sp. | molecular biology | oral cavity |
| Methanobacterium congolense | molecular biology | stool |
| Methanoculleus chikugoensis | molecular biology | stool |
| Methanobrevibacter millerae | molecular biology | stool |
| Methanobrevibacter massiliense | molecular biology | oral cavity |
| Candidatus Nitrososphaera | molecular biology | oral cavity |
| evergladensis | | |
| Methanoculleus bourgensis | molecular biology | oral cavity |

FIG. 2A shows the detection of archaea 16S ribosomal RNA PCR products (vaginosis samples) by 1.5% agarose gel electrophoresis. Lanes E.V1 to E.V11: vaginosis samples; T−: negative control; and MT: size marker.

FIG. 2B shows the detection of PCR products targeting the methanogenic mcrA gene (vaginosis samples) by 1.5% agarose gel electrophoresis. Lanes E.1 to E.12: vaginosis samples; T−: negative control; and MT: size marker.

TABLE 1
Detection of the presence of M. smithii by standard PCR
targeting the 16S rRNA and mcrA genes for 77 samples.

| Sample no. | Sample nature | M. smithii 16S r PCR | mcrA PCR |
|---|---|---|---|
| 1 | FN | NEGATIVE | NEGATIVE |
| 2 | FN | NEGATIVE | NEGATIVE |
| 3 | FN | NEGATIVE | NEGATIVE |
| 4 | FN | NEGATIVE | NEGATIVE |
| 5 | FN | NEGATIVE | NEGATIVE |
| 6 | FN | NEGATIVE | NEGATIVE |
| 7 | FN | NEGATIVE | NEGATIVE |
| 8 | FN | NEGATIVE | NEGATIVE |
| 9 | FN | NEGATIVE | NEGATIVE |
| 10 | FN | NEGATIVE | NEGATIVE |
| 11 | FN | NEGATIVE | NEGATIVE |
| 12 | FN | NEGATIVE | NEGATIVE |
| 13 | FN | NEGATIVE | NEGATIVE |
| 14 | FN | NEGATIVE | NEGATIVE |
| 15 | FN | NEGATIVE | NEGATIVE |
| 16 | FN | NEGATIVE | NEGATIVE |
| 17 | FN | NEGATIVE | NEGATIVE |
| 18 | FN | NEGATIVE | NEGATIVE |
| 19 | FN | NEGATIVE | NEGATIVE |
| 20 | FN | NEGATIVE | NEGATIVE |
| 21 | FN | NEGATIVE | NEGATIVE |
| 22 | FN | NEGATIVE | NEGATIVE |
| 23 | FN | NEGATIVE | NEGATIVE |
| 24 | FN | NEGATIVE | NEGATIVE |
| 25 | FN | NEGATIVE | NEGATIVE |
| 26 | FN | NEGATIVE | NEGATIVE |
| 27 | FN | NEGATIVE | NEGATIVE |
| 28 | FN | NEGATIVE | NEGATIVE |
| 29 | FN | NEGATIVE | NEGATIVE |
| 30 | FN | NEGATIVE | NEGATIVE |
| 31 | FN | NEGATIVE | NEGATIVE |
| 32 | FN | NEGATIVE | NEGATIVE |
| 33 | FN | NEGATIVE | NEGATIVE |
| 34 | ND | NEGATIVE | NEGATIVE |
| 35 | FN | NEGATIVE | NEGATIVE |
| 36 | VB | POSITIVE | POSITIVE |
| 37 | VB | POSITIVE | POSITIVE |
| 38 | VB | POSITIVE | POSITIVE |
| 39 | VB | POSITIVE | POSITIVE |
| 40 | VB | POSITIVE | POSITIVE |
| 41 | VB | POSITIVE | POSITIVE |
| 42 | VB | POSITIVE | POSITIVE |
| 43 | VB | POSITIVE | POSITIVE |
| 44 | VB | POSITIVE | POSITIVE |
| 45 | VB | POSITIVE | POSITIVE |
| 46 | VB | POSITIVE | POSITIVE |
| 47 | VB | POSITIVE | POSITIVE |
| 48 | VB | POSITIVE | POSITIVE |
| 49 | VB | POSITIVE | POSITIVE |
| 50 | VB | POSITIVE | POSITIVE |
| 51 | FN | NEGATIVE | NEGATIVE |
| 52 | FN | NEGATIVE | NEGATIVE |
| 53 | FN | NEGATIVE | NEGATIVE |
| 54 | FN | NEGATIVE | NEGATIVE |
| 55 | FN | NEGATIVE | NEGATIVE |
| 56 | FN | NEGATIVE | NEGATIVE |
| 57 | FN | NEGATIVE | NEGATIVE |
| 58 | FN | NEGATIVE | NEGATIVE |
| 59 | FN | NEGATIVE | NEGATIVE |
| 60 | FN | NEGATIVE | NEGATIVE |
| 61 | FN | NEGATIVE | NEGATIVE |
| 62 | FN | NEGATIVE | NEGATIVE |
| 63 | FN | NEGATIVE | NEGATIVE |
| 64 | FN | NEGATIVE | NEGATIVE |
| 65 | FN | NEGATIVE | NEGATIVE |
| 66 | FN | NEGATIVE | NEGATIVE |
| 67 | FN | NEGATIVE | NEGATIVE |
| 68 | FN | NEGATIVE | NEGATIVE |
| 69 | FN | NEGATIVE | NEGATIVE |
| 70 | FN | NEGATIVE | NEGATIVE |
| 71 | FN | NEGATIVE | NEGATIVE |
| 72 | FN | NEGATIVE | NEGATIVE |
| 73 | FN | NEGATIVE | NEGATIVE |
| 74 | FN | NEGATIVE | NEGATIVE |
| 75 | FN | NEGATIVE | NEGATIVE |
| 76 | FN | NEGATIVE | NEGATIVE |
| 77 | FN | NEGATIVE | NEGATIVE |

FN = normal flora;
BV = bacterial vaginosis.

### EXAMPLE 4: DETECTION OF METHANOBREVIBACTER SMITHII IN PATIENT VAGINAL SPECIMENS BY AMPLIFICATION AND DETECTION BY PROBE BY REAL-TIME PCR

In this example, the same collection of vaginal specimens tested in Example 3 were tested for Methanobrevibacter smithii by real-time PCR targeting the 16S rRNA, mcrA and rpoB genes.

The molecular detection of the methanogenic archaea Methanobrevibacter smithii was performed by real-time PCR after extraction of the DNA according to a protocol as described in Example 3 with the following primers and probes.

| Microorganisms | Targets | Nucleotide sequences |
|---|---|---|
| M. smithii | 16S rRNA | Sense primer: |
| | | SEQ ID NO: 5: CCGGGTATCTAATCCGGTTC |
| | | Antisense primer: |
| | | SEQ ID NO: 6: |
| | | CTCCCAGGGTAGAGGTGAAA |
| | | Probe: |
| | | SEQ ID NO: 7: |
| | | FAM-CCGTCAGAATCGTTCCAGTCAG-TAMRA |
| M. smithii | mcrA gene | Sense primer: |
| | | SEQ ID NO: 8: |
| | | GCTCTACGACCAGATMTGGCTTGG |
| | | Antisense primer: |
| | | SEQ ID NO: 9: |
| | | CCGTAGTACGTGAAGTCATCCAGCA |
| | | Probe: |
| | | SEQ ID NO: 10: |
| | | FAM-ARGCACCKAACAMCATGGACACWGT- |
| | | TAMRA |
| M. smithii | rpoB gene | Sense primer: |
| | | SEQ ID NO: 11: |
| | | AAGGGATTTGCACCCAACAC |
| | | Antisense primer: |
| | | SEQ ID NO: 12: |
| | | GACCACAGTTAGGACCCTCTGG |
| | | Probe: |
| | | SEQ ID NO: 13: |
| | | FAM- |
| | | ATTTGGTAAGATTTGTCCGAATG-TAMRA |
| Human albumin | Exon 12 | Sense primer: |
| | | SEQ ID NO: 14: |
| | | GCTGTCATCTCTTGTGGGCTGT |
| | | Antisense primer: |
| | | SEQ ID NO: 15 |
| | | AAACTCATGGGAGCTGCTGGTTC |
| | | Probe: |
| | | SEQ ID NO: 16 |
| | | FAM- |
| | | CCTGTCATGCCCACACAAATCTCTCC-TAMRA |

After DNA extraction, a set of real-time polymerase chain reactions were performed as follows. A 20 μL mix (reaction mixture) was prepared with 10 μL of mix (Thermo Fisher Scientific, Villebon sur Yvette, France); distilled water (Sigma Aldrich, Saint-Quentin-Fallavier, France), a 5 μM probe; a sense (5′) primer 20 μM 0.5 μL; an antisense (3′) primer 20 μM 0.5 μL, uracil DNA glycosylase 0.5 μL and 5 μL extracted DNA. PCR reactions are performed in the Stratagene MX3000P apparatus (BIO-RAD, Marnes-la-Coquette, France) according to the following program: 50° C. for 2 minutes, 95° C. for 5 minutes, 02 steps at 39 cycles (95° C. for 5 seconds and 60° C. for 30 seconds).

All negative controls tested are negative in real-time PCR. Of the 77 specimens tested, 62 specimens corresponding to samples from normal vaginal flora are negative in real-time PCR (Ct≥39) and 15 specimens corresponding to samples from vaginosis are positive in real-time PCR (Ct<39) (Table 2). “CT”, a measure of the number of cycles that provide a positive result, the smaller the CT, the greater the amount of amplified DNA.

TABLE 2
Detection of the presence of M. smithii by real-time PCR
targeting the 16S RNA ribosomal gene for 77 samples.

| | Sample | | |
|---|---|---|---|
| Sample no. | nature | Ct | Interpretation |
| 1 | FN | N/A | NEGATIVE |
| 2 | FN | N/A | NEGATIVE |
| 3 | FN | N/A | NEGATIVE |
| 4 | FN | N/A | NEGATIVE |
| 5 | FN | N/A | NEGATIVE |
| 6 | FN | N/A | NEGATIVE |
| 7 | FN | N/A | NEGATIVE |
| 8 | FN | N/A | NEGATIVE |
| 9 | FN | N/A | NEGATIVE |
| 10 | FN | N/A | NEGATIVE |
| 11 | FN | N/A | NEGATIVE |
| 12 | FN | N/A | NEGATIVE |
| 13 | FN | N/A | NEGATIVE |
| 14 | FN | N/A | NEGATIVE |
| 15 | FN | N/A | NEGATIVE |
| 16 | FN | N/A | NEGATIVE |
| 17 | FN | N/A | NEGATIVE |
| 18 | FN | N/A | NEGATIVE |
| 19 | FN | N/A | NEGATIVE |
| 20 | FN | N/A | NEGATIVE |
| 21 | FN | N/A | NEGATIVE |
| 22 | FN | N/A | NEGATIVE |
| 23 | FN | N/A | NEGATIVE |
| 24 | FN | N/A | NEGATIVE |
| 25 | FN | N/A | NEGATIVE |
| 26 | FN | N/A | NEGATIVE |
| 27 | FN | N/A | NEGATIVE |
| 28 | FN | N/A | NEGATIVE |
| 29 | FN | N/A | NEGATIVE |
| 30 | FN | N/A | NEGATIVE |
| 31 | FN | N/A | NEGATIVE |
| 32 | FN | N/A | NEGATIVE |
| 33 | FN | N/A | NEGATIVE |
| 34 | FN | N/A | NEGATIVE |
| 35 | FN | N/A | NEGATIVE |
| 36 | VB | 37.73 | POSITIVE |
| 37 | VB | 36.34 | POSITIVE |
| 38 | VB | 29.56 | POSITIVE |
| 39 | VB | 28.03 | POSITIVE |
| 40 | VB | 29.87 | POSITIVE |
| 41 | VB | 31.54 | POSITIVE |
| 42 | VB | 27.70 | POSITIVE |
| 43 | VB | 29.79 | POSITIVE |
| 44 | VB | 30.06 | POSITIVE |
| 45 | VB | 29.98 | POSITIVE |
| 46 | VB | 32.86 | POSITIVE |
| 47 | VB | 29.57 | POSITIVE |
| 48 | VB | 28.06 | POSITIVE |
| 49 | VB | 32.78 | POSITIVE |
| 50 | VB | 34.85 | POSITIVE |
| 51 | FN | N/A | NEGATIVE |
| 52 | FN | N/A | NEGATIVE |
| 53 | FN | N/A | NEGATIVE |
| 54 | FN | N/A | NEGATIVE |
| 55 | FN | N/A | NEGATIVE |
| 56 | FN | N/A | NEGATIVE |
| 57 | FN | N/A | NEGATIVE |
| 58 | FN | N/A | NEGATIVE |
| 59 | FN | N/A | NEGATIVE |
| 60 | FN | N/A | NEGATIVE |
| 61 | FN | N/A | NEGATIVE |
| 62 | FN | N/A | NEGATIVE |
| 63 | FN | N/A | NEGATIVE |
| 64 | FN | N/A | NEGATIVE |
| 65 | FN | N/A | NEGATIVE |
| 66 | FN | N/A | NEGATIVE |
| 67 | FN | N/A | NEGATIVE |
| 68 | FN | N/A | NEGATIVE |
| 69 | FN | N/A | NEGATIVE |
| 70 | FN | N/A | NEGATIVE |
| 71 | FN | N/A | NEGATIVE |
| 72 | FN | N/A | NEGATIVE |
| 73 | FN | N/A | NEGATIVE |
| 74 | FN | N/A | NEGATIVE |
| 75 | FN | N/A | NEGATIVE |
| 76 | FN | N/A | NEGATIVE |
| 77 | FN | N/A | NEGATIVE |

FN = normal flora;
BV = bacterial vaginosis.
N/A = not amplified (Ct ≥ 39),
Ct = threshold cycle.

The results according to the present invention demonstrate that the detection of Methanobrevibacter smithii is perfectly discriminatory for the diagnosis of bacterial vaginosis.

The novelty of the present invention is to be able to propose for the first time a simple tool for the diagnosis of BV based on the molecular detection of Methanobrevibacter smithii.

Furthermore, the specificity of the molecular detection described here leads to the understanding that the detection of one or more antigens specific to M. smithii by any appropriate method, as well as the detection of the presence of methane in the vaginal cavity or from a sample of the vaginal cavity by any appropriate method are also methods for the diagnosis of bacterial vaginosis, could constitute a simplex and non-quantitative diagnostic test for the presence of M. smithii in a sample from the vaginal cavity falling within the scope of the present invention, in particular a method for detecting one or more antigens specific to M. smithii, in particular by immunodetection using specific antibodies or a colorimetric method.

It is also understood that this method for diagnosing BV allows follow-up for the evaluation and therapeutic management of BV during pregnancy.

### BIBLIOGRAPHY

Amsel R, Totten P A, Spiegel C A, Chen K C, Eschenbach D, Holmes K K. Nonspecific vaginitis. Diagnostic criteria and microbial and epidemiologic associations. Am J Med. 1983; 74:14-22.
Belay N, Mukhopadhyay B, Conway de Macario E, Galask R, Daniels L. Methanogenic bacteria in human vaginal samples. J Clin Microbiol. 1990; 28:1666-8.
Bradshaw C S, Morton A N, Hocking J. High recurrence rates of bacterial vaginosis over the course of 12 months after oral metronidazole therapy and factors associated with recurrence. J Infect Dis 2006; 193:1478-86.
Bradshaw C S, Tabrizi S N, Fairley C K, Morton A N, Rudland E, Garland S M. The association of Atopobium vaginae and Gardnerella vaginalis with bacterial vaginosis and recurrence after oral metronidazole therapy. J Infect Dis. 2006; 194:828-36.
Bretelle F, Rozenberg P, Pascal A, Favre R, Bohec C, Loundou A, Senat M V, Aissi G, Lesavre N, Brunet J, Heckenroth H, Luton D, Raoult D, Fenollar F; Groupe de Recherche en Obstetrique Gynecologie. High Atopobium vaginae and Gardnerella vaginalis vaginal loads are associated with preterm birth. Clin Infect Dis. 2015; 60:860-7.
Drancourt M, Nkamga V D, Lakhe N A, Régis J M, Dufour H, Fournier P E, Bechah Y, Scheid W M, Raoult D. Evidence of Archaeal Methanogens in Brain Abscess. Clin Infect Dis. 2017 Jul. 1; 65(1):1-5.
Drancourt M, Michel-Lepage A, Boyer S, Raoult D. The Point-of-Care Laboratory in Clinical Microbiology. Clin Microbiol Rev. 2016 Jul. 1; 29(3):429-47.
Dridi B, Henry M, El Khéchine A, Raoult D, Drancourt M (2009). High prevalence of Methanobrevibacter smithii and Methanosphaera stadtmanae detected in the human gut using an improved DNA detection protocol. PLoS One 4(9):e7063.
Ferris M J, Masztal A, Aldridge K E, Fortenberry J D, Fidel P L Jr, Martin D H. Association of Atopobium vaginae, a recently described metronidazole resistant anaerobe, with bacterial vaginosis. BMC Infect Dis. 2004 Feb. 13; 4:5.
Ferris, M. J., A. Masztal, and D. H. Martin. Use of species-directed 16S rRNA gene PCR primers for detection of Atopobium vaginae in patients with bacterial vaginosis. J Clin Microbiol. 2004; 42:5892-4.
Fredricks D N, Fiedler T L, Marrazzo J M. Molecular identification of bacteria associated with bacterial vaginosis. N Engl J Med. 2005; 353:1899-911.
Guise J M, Mahon S M, Aickin M, Helfand M, Peipert J F, Westhoff C. Screening for bacterial vaginosis in pregnancy. Am J Prev Med. 2001; 20(3 Suppl):62-72.
Hauth J C, Goldenberg R L, Andrews W W, DuBard M B, Copper R L. Reduced incidence of preterm delivery with metronidazole and erythromycin in women with bacterial vaginosis. N Engl J Med. 1995; 333:1732-6.
Hebb J K, Cohen C R, Astete S G, Bukusi E A, Totten P A. Detection of novel organisms associated with salpingitis, by use of 16S rDNA polymerase chain reaction. J Infect Dis. 2004; 190:2109-20.
Ison C A, Hay P E. Validation of a simplified grading of Gram stained vaginal smears for use in genitourinary medicine clinics. Sex Transm Infect. 2002; 78:413-5.
Khelaifia S, Lagier J C, Nkamga V D, Guilhot E, Drancourt M, Raoult D. Aerobic culture of methanogenic archaea without an external source of hydrogen. Eur J Clin Microbiol Infect Dis. 2016 June; 35(6):985-91.
Menard J P, Mazouni C, Fenollar F, Raoult D, Boubli L, Bretelle F. Diagnosticvaccuracy of quantitative real-time PCR assay versus clinical and Gram stainvidentification of bacterial vaginosis. Eur J Clin Microbiol Infect Dis. 2010; 29:1547-52;
Menard J P, Mazouni C, Salem-Cherif I, Fenollar F, Raoult D, Boubli L, Gamerre M, Bretelle F. High vaginal concentrations of Atopobium vaginae and Gardnerella vaginalis in women undergoing preterm labor. Obstet Gynecol. 2010; 115:134-40.
Menard J P, Fenollar F, Henry M, Bretelle F, Raoult D. Molecular quantification of Gardnerella vaginalis and Atopobium vaginae loads to predict bacterial vaginosis. Clin Infect Dis. 2008; 47:33-43.
Nkamga V D, Lotte R, Roger P M, Drancourt M, Ruimy R. Methanobrevibacter smithii and Bacteroides thetaiotaomicron cultivated from a chronic paravertebral muscle abscess. Clin Microbiol Infect. 2016 December; 22(12):1008-1009.
Nugent R P, Krohn M A, Hillier S L. Reliability of diagnosing bacterial vaginosis is improved by a standardized method of gram stain interpretation. J Clin Microbiol. 1991; 29:297-301.
Rodriguez Jovita M, Collins M D, Sjoden B, Falsen E. Characterization of a novel Atopobium isolate from the human vagina: description of Atopobium vaginae sp. nov. Int J Syst Bacteriol. 1999; 49:1573-6.
Schwebke J R, Lawing L F. Prevalence of Mobiluncus spp among women with and without bacterial vaginosis as detected by polymerase chain reaction. Sex Transm Dis. 2001; 28:195-9.
Sha B E, Chen H Y, Wang Q J, Zariffard M R, Cohen M H, Spear G T. Utility of Amsel criteria, Nugent score, and quantitative PCR for Gardnerella vaginalis, Mycoplasma hominis, and Lactobacillus sp. for diagnosis of bacterial vaginosis in human immunodeficiency virus-infected women. J Clin Microbiol. 2005; 43:4607-12.
Spiegel C A., Bacterial Vaginosis. Clinical Microbiology Reviews. 1991; 4:485-502.
Thomason J L, Anderson R J, Gelbart S M, Osypowski P J, Scaglione N J, el Tabbakh G, James J A. Simplified gram stain interpretive method for diagnosis of bacterial vaginosis. Am J Obstet Gynecol. 1992; 167:16-9.
Thorsen P, Jensen I P, Jeune B, Ebbesen N, Arpi M, Bremmelgaard A, Moller B R. Few microorganisms associated with bacterial vaginosis may constitute the pathologic core: a population-based microbiologic study among 3596 pregnant women. Am J Obstet Gynecol. 1998; 178:580-7.
Verhelst, R., H. Verstraelen, G. Claeys, G. Verschraegen, J. Delanghe, L. Van Simaey, C. De Ganck, M. Temmerman, and M. Vaneechoutte. 2004. Cloning of 16S rRNA genes amplified from normal and disturbed vaginal microflora suggests a strong association between Atopobium vaginae, Gardnerella vaginalis and bacterial vaginosis. BMC. Microbiol 2004; 4:16.
Verhelst R, Verstraelen H, Claeys G, Verschraegen G, Van Simaey L, De Ganck C, De Backer E, Temmerman M, Vaneechoutte M. Comparison between Gram stain and culture for the characterization of vaginal microflora: definition of a distinct grade that resembles grade I microflora and revised categorization of grade I microflora. BMC Microbiol. 2005 Oct. 14; 5:61. Vianna M E, Conrads G, Gomes B P, et al. T-RFLP-basedmcrA gene analysis of methanogenic archaea in association with oral infections and evidence of a novel Methanobrevibacter phylotype. Oral Microbiology and Immunology, 24(5), pp. 417-422.

1. A method for the in vitro diagnosis of the presence of bacterial vaginosis in a patient, characterized in that the following steps are carried out wherein:
a) the total DNA contained in the vaginal secretion sample is extracted by a method capable of extracting DNA from methanogenic archaea, preferably comprising at least two steps of enzymatic and/or mechanical DNA lysis, and
b) the presence of bacterial vaginosis is determined if the DNA extracted from said vaginal secretion sample of said patient is detected to contain said sequence specific to human DNA and at least one nucleic acid sequence specific to said methanogenic archaea Methanobrevibacter smithii, the concentrations of the DNA fragments of said sequence specific to human DNA and said sequence specific to Methanobrevibacter smithii in said vaginal secretion sample each being greater than or equal to 10¹ copies/mL.

2. The method as claimed in claim 1, characterized in that the presence of Methanobrevibacter smithii is determined by detecting the presence of at least one nucleic acid sequence specific to said Methanobrevibacter smithii methanogenic archaea present in a single copy in said Methanobrevibacter smithii archaea, said sequence specific to Methanobrevibacter smithii having a size of less than 150 nucleotides.

3. The method as claimed in claim 1, characterized in that step b), comprises the following steps wherein:
b.1) PCR-type enzymatic amplification of at least one said sequence specific to said methanogenic archaea Methanobrevibacter smithii is performed in the DNA extracted from said vaginal secretion sample, and
b.2) amplified fragments of said sequence specific to Methanobrevibacter smithii are detected preferably by sequencing, by agarose gel electrophoresis or by using labeled probes specific for said sequence specific to said Methanobrevibacter smithii methanogenic archaea of sequences distinct from those of said amplification primers.

4. The method as claimed in claim 2, characterized in that reactions are carried out to amplify and quantify a sequence specific to human DNA in the DNA extracted from the test sample, comprising a sequence specific to human albumin.

5. The method as claimed in claim 2, characterized in that said sequence specific to said methanogenic archaea Methanobrevibacter smithii comprises or is comprised in one of the following fragments:
the fragment from positions 334632 to 334687 of the 16S ribosomal RNA gene with GenBank accession number NC 009515.1;
the fragment from positions 1734995 to 1735069 of the mcrA gene of the protein methyl coenzyme M reductase alpha subunit of 157 amino acids and 16.942 Da with GenBank accession number AAW80308.1;
the fragment from positions 876305 to 876370 of the rpoB gene of the M. smithii RNA polymerase subunit B protein of 186 amino acids and 20.836 Da with GenBank accession number ABYV02000000.

6. The method as claimed in claim 4, characterized in that said sequence specific to human DNA in the test sample comprises the fragment from positions 16283-16423 of exon 12 of the human albumin gene with GenBank accession number M12523.1.

7. The method as claimed in claim 3, characterized in that in step b) the following steps are carried out wherein:
b.1) a PCR-type enzymatic amplification reaction is carried out on the DNA of at least one said sequence specific to said methanogenic archaea Methanobrevibacter smithii, in the DNA extracted from said samples to be tested, using at least one set of primers capable of amplifying said sequence specific to said methanogenic archaea Methanobrevibacter smithii, and
b.2) it is checked whether the possible amplifiers of the DNA extracted from said samples to be tested comprise a said specific sequence using a hydrolysis probe comprising a sequence specific to said sequence specific to Methanobrevibacter smithii and flanked by the sequences of said primers.

8. The method as claimed in claim 7, characterized in that co-amplification and quantification reactions are carried out by using two sets of primers and hydrolysis probes specific, respectively, on the one hand, to said sequence specific to the archaea Methanobrevibacter smithii, and, on the other hand, to a sequence specific to human DNA in the sample to be tested, preferably a sequence specific to human albumin, and said sequence specific to human DNA comprising a sequence of said probe flanked by sequences suitable for use as said primers in a PCR-type amplification reaction of said sequence specific to human DNA.

9. The method as claimed in claim 1, characterized in that the presence of bacterial vaginosis is determined if, in the DNA extracted from a patient vaginal secretion sample, the following two conditions a) and b) are met:
a) the concentration Ca of the human albumin DNA fragment is greater than or equal to 10¹ copies/mL, and
b) the concentration Cm of said DNA fragment specific to Methanobrevibacter smithii is greater than or equal to 10¹ copies/mL.

10. The method as claimed in claim 3, characterized in that in step a), a method of extracting the total DNA contained in the vaginal secretion sample including the extraction of DNA from Methanobrevibacter smithii is carried out, comprising the following steps wherein:
a.1) a step of mechanical lysis, preferably sonication, of the vaginal secretion sample is carried out, and
a.2) enzymatic lysis of said sample is carried out.

11. The method as claimed in claim 3, characterized in that said sequence specific to said methanogenic archaea Methanobrevibacter smithii is selected from the following sequences including probe sequences (underlined) flanked by primer sequences (in bold) or their reverse and complementary sequences for antisense primers:
-for the 16S rRNA sequence;
SEQ ID NO: 2 =
5′CCGGGTATCTAATCCGGTTCCCGTCAGAATCGTTCC
AGTCAGCTCCCAGGGTAGAGGTGAAA-3′;
-for the mcrA gene sequence;
SEQ ID NO: 3 =
5′-GCTCTACGACCAGATMTGGCTTGGARGCACCKAA
CAMCATGGACACWGTCCGTAGTACGTGAAGTCATCCAGCA-3′;
and
-for the rpoB sequence;
SEQ ID NO: 4 =
5′-AAGGGATTTGCACCCAACACATTTGGTAAGATTTGTCCGAATG
GACCACAGTTAGGACCCTCTGG-3′.

12. The method as claimed in claim 8, characterized in that said sequence of exon 12 of the human albumin gene specific to human DNA in the test sample comprises the following sequence listing sequence or the complementary sequence:
SEQ ID NO: 1 =
5′-GCTGTCATCTCTTGTGGGCTGTAATCATCGTTTAAGAGTAATAT
TGCAAAACCTGTCATGCCCACACAAATCTCTCCCTGGCATTGTTG
TCTTTGCAGATGTCAGTGAAAGAGAACCAGCAGCTCCCATGAGTTT-3′.

13. The method as claimed in claim 11, characterized in that the sets of primers and preferably probes are used, which are if need be selected from the following sequences of the sequence listing appended to the present description, or their complementary sequences:
-For the 16S rRNA gene:
5′ primer:
SEQ ID NO: 5 =
5′-CCGGGTATCTAATCCGGTTC-3′
3′ primer:
SEQ ID NO: 6 =
5′-CTCCCAGGGTAGAGGTGAAA-3′
-For the mcrA gene:
5′ primer:
SEQ ID NO: 8 =
5′-GCTCTACGACCAGATMTGGCTTGG-3′
3′ primer:
SEQ ID NO: 9 =
5′-CCGTAGTACGTGAAGTCATCCAGCA-3′.
-For the rpoB gene:
5′ primer:
SEQ ID NO: 11 =
5′-AAGGGATTTGCACCCAACAC-3′
3′ primer:
SEQ ID NO: 12 =
5′-GACCACAGTTAGGACCCTCTGG-3′
-For human albumin:
5′ primer:
SEQ ID NO: 14 =
5′-GCTGTCATCTCTTGTGGGCTGT-3′
3′ primer:
SEQ ID NO: 15 =
3′-AAACTCATGGGAGCTGCTGGTTC-3′

14. A diagnostic kit for carrying out a diagnostic method as claimed in claim 1, characterized in that it comprises at least:
reagents for extracting the total DNA contained in a vaginal secretion sample including DNA from Methanobrevibacter smithii, as well as
a set of primers specific for at least one DNA sequence specific to said methanogenic archaea Methanobrevibacter smithii and preferably at least one probe specific for said sequence specific to said methanogenic archaea Methanobrevibacter smithii, and
reagents for carrying out a PCR-type DNA amplification reaction.

15. The diagnostic kit as claimed in claim 14, comprising:
at least one set of primers and probes of sequences selected from the 3 sets of sequences SEQ ID NO: 5 to 7, SEQ ID NO: 8 to 10 and SEQ ID NO: 11 to 13, and
at least one set of primers and probe of sequences SEQ ID NO: 14 to 16.
